(19) United States
(12) Patent Application Publication
(10) Pub. No.: US 2026/0259226 A1
(43) Pub. Date: Sep. 3, 2026

(54) DLL1 MARKER PANELS FOR EARLY DETECTION OF SEPSIS

(71) Applicant: Roche Diagnostics Operations, Inc., Indianapolis, IN (US)

(72) Inventors: Felix Gruenewald, Penzberg (DE); Kathrin Heinz, München (DE); Martin Klammer, Muenchen (DE); Anna Maria Plaszczyca, München (DE); Philipp Schuetz, Basel (CH); Maria von Holtey, Basel (CH); Stephen Weber, München (DE)

(21) Appl. No.: 18/840,264

(22) PCT Filed: Feb. 20, 2023
(86) PCT No.: PCT/EP2023/054199

§ 371 Date: Aug. 21, 2024

(30) Foreign Application Priority Data
Feb. 21, 2022 (EP) 22157786.9

(51) Int. Cl. G01N 33/68

(52) U.S. Cl. CPC G01N 33/6893; G01N 2333/475; G01N 2333/575; G01N 2333/705; G01N 2333/8139; G01N 2333/91205; G01N 2800/26; G01N 2800/50

(57) ABSTRACT

The present invention concerns the field of diagnostics. Specifically, it relates to a method for assessing a subject with suspected infection comprising the steps of determining the amount of a first biomarker in a sample of the subject, said first biomarker being DLL1, determining the amount of a second biomarker in a sample of the subject, said second biomarker being GDF15, comparing the amounts of the biomarkers to references for said biomarkers and/or calculating a score for assessing the subject with suspected infection based on the amounts of the biomarkers, and assessing said subject based on the comparison and/or the calculation. The invention also relates to the use of a first biomarker being DLL1 and a second biomarker being GDF15, or a detection agent specifically binding to said first biomarker and a detection agent specifically binding to said second biomarker for assessing a subject with suspected infection. Moreover, the invention further relates to a computer-implemented method for assessing a subject with suspected infection and a device and a kit for assessing a subject with suspected infection.

The present invention concerns the field of diagnostics. Specifically, it relates to a method for assessing a subject with suspected infection comprising the steps of determining the amount of a first biomarker in a sample of the subject, said first biomarker being DLL1, determining the amount of a second biomarker in a sample of the subject, said second biomarker being GDF15, comparing the amounts of the biomarkers to references for said biomarkers and/or calculating a score for assessing the subject with suspected infection based on the amounts of the biomarkers, and assessing said subject based on the comparison and/or the calculation. The invention also relates to the use of a first biomarker being DLL1 and a second biomarker being GDF15, or a detection agent specifically binding to said first biomarker and a detection agent specifically binding to said second biomarker for assessing a subject with suspected infection. Moreover, the invention further relates to a computer-implemented method for assessing a subject with suspected infection and a device and a kit for assessing a subject with suspected infection.

Infection, in particular, infection occurring in patients having more severe signs and symptoms thereof such as those presenting in emergency units, may sometimes develop to more life threatening medical conditions including systemic inflammatory response syndrome (SIRS) and sepsis.

According to the Sepsis-3 definition, sepsis is defined as a life threatening organ dysfunction caused by a dysregulated host response to infection. As it develops rapidly, early recognition is important for sepsis patient management and start of correct therapeutic measures including appropriate antibiotic therapy within the first hour of admission, and start of resuscitation with intravenous fluids and vasoactive drugs (surviving sepsis campaign guidelines 2016). Delay for every hour, incrementally increases morbidity and mortality.

Diagnosis of sepsis is based on clinical signs and symptoms that are non-specific and can be easily missed. Thus, patients are frequently misdiagnosed and the severity of disease is often underestimated. There is no gold standard for diagnosis of sepsis in general and in the emergency department in particular so far. In high income countries c-reactive protein (CRP), Procalcitonin (PCT) and white blood cell (WBC) count are often used in emergency units for detection of patients with bloodstream infection at risk for development of sepsis, together with lactate for detection of septic shock. In low income countries, diagnosis is mostly based on clinical signs and symptoms and in some instances SIRS and SOFA criteria. However, in the most current guidelines, besides lactate, no biomarker has been listed to diagnose sepsis (with the exception of clinical chemistry, BGE and hematology components of the SOFA score). PCT has only been recommended to potentially deescalate antibiotic therapy, however, with moderate evidence. Limitations of PCT in sepsis diagnosis are mainly the moderate sensitivity and specificity.

WO 2007/009071 discloses method of diagnosing an inflammatory response in a test subject based on sFlt-1. The disclosed method further comprises analyzing the level of at least one of VEGF, PIGF5, TNF-α, IL-6, D-dimer, P-selectin, ICAM-I, VCAM-I, Cox-2, or PAI-I.

EP 2 174 143 B1 discloses an in vitro method for prognosis for a patient having a primary disease not being an infection, the method comprising determining the level of procalcitonin.

A multitude of markers have been suggested to be useful for detection or diagnosis of sepsis.

These include, amongst many others, PCT, Presepsin, GDF-15, sFLT, inflammatory markers like CRP or interleukins, or markers specific of organ failure (see, e.g., Spanuth, 2014, Comparison of sCD14-ST (presepsin) with eight biomarkers for mortality prediction in patients admitted with acute heart failure, 2014 AACC Annual Meeting Abstracts. B-331; van Engelen, 2018, Crit Care Clin 34 (1): 139-152.)

WO2015/031996 describes biomarkers for early determination of a critical or life threatening response to illness and/or treatment response.

Delta-like protein 1 (DLL1, Uniprot accession ID 000548) is a transmembrane cell surface protein consisting of 723 amino acids. DLL1 is one of the four canonical ligand of Notch receptors and binds the extracellular domain of Notch receptor. The Notch signaling pathway regulates many aspects of embryonic development, as well as differentiation processes and tissue homeostasis in multiple adult organ systems, including hematopoietic and immune systems. Upon interaction between a Notch receptor and DLL1, both the receptor and its ligand are cleaved from the surface, resulting in the generation of soluble DLL1 (sDLL1).

Expression of DLL1 is increased in human monocytes and in a mouse model upon lipopolysaccharide (LPS) stimulation or bacterial infection. Induction of DLL1 expression is mediated by Toll-Like Receptor (TLR) signaling indirectly Through Cytokine Receptor-Triggered STAT3 Activation. (Hildebrand et al., Front. Cell. Infect. Microbiol. 8:241. doi: 10.3389/fcimb.2018.00241 (2018)).

Plasma concentrations of DLL1 were reported to be elevated in two cohorts of patients with sepsis or septic shock, when compared to healthy control patients or control patients having underwent cisceral surgery (Hildebrand et al., Front. Cell. Infect. Microbiol. 9:267. (2019); doi: 10.3389/fcimb.2019.00267). Decker et al. reported that DLL1 levels were higher in patients with bacterial infection vs no infection after liver transplantation. 93 patients were analyzed (Decker et al., Diagnostics (Basel). 2020 Oct. 31;10 (11): 894. doi: 10.3390/diagnostics10110894. PMID: 33142943; PMCID: PMC7693674.

Norum et al. reported that DLL1 plasma levels were elevated in heart transplant recipients (Norum et al., Am J Transplant. 2019 April;19 (4): 1050-1060. doi: 10.1111/ajt.15141. Epub 2018 Nov. 5. PMID: 30312541.

EP 3 701 268 B1 discloses the use of delta-like ligand 1 protein or a nucleotide sequence coding for delta-like ligand 1 protein as a biomarker for the in vitro diagnosis of a severe infection.

However, there is still a need for biomarkers, which allow for a reliable and early assessment of patients exhibiting signs and symptoms of infection.

The present invention, therefore, provides means and methods complying with these needs.

The present invention relates to a method for assessing a subject with suspected infection comprising the steps of:
(a) determining the amount of a first biomarker in a sample of the subject, said first biomarker being DLL1 (Delta-like protein 1);
(b) determining the amount of a second biomarker in a sample of the subject, said second biomarker being GDF15 (Growth-Differentiation Factor-15);
(c) comparing the amounts of the biomarkers to references for said biomarkers and/or calculating a score for assessing the subject with suspected infection based on the amounts of the biomarkers; and
(d) assessing said subject based on the comparison and/or the calculation made in step (c).

It is to be understood that as used in the specification and in the claims, “a” or “an” can mean one or more, depending upon the context in which it is used. Thus, for example, reference to “an” item can mean that at least one item can be utilized.

As used in the following, the terms “have”, “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B”, “A comprises B” and “A includes B” may both refer to a situation in which, besides B, no other element is present in A (i.e. a situation in which A solely and exclusively consists of B) and to a situation in which, besides B, one or more further elements are present in entity A, such as element C, elements C and D or even further elements. The term “comprising” also encompasses embodiments where only the items referred to are present, i.e. it has a limiting meaning in the sense of “consisting of′.

Further, as used in the following, the terms “particularly”, “more particularly”, “typically”, and “more typically” or similar terms are used in conjunction with additional/alternative features, without restricting alternative possibilities. Thus, features introduced by these terms are additional/alternative features and are not intended to restrict the scope of the claims in any way. The invention may, as the skilled person will recognize, be performed by using alternative features. Similarly, features introduced by “in an embodiment of the invention” or similar expressions are intended to be additional/alternative features, without any restriction regarding alternative embodiments of the invention, without any restrictions regarding the scope of the invention and without any restriction regarding the possibility of combining the features introduced in such way with other additional/alternative or non-additional/alternative features of the invention.

Further, it will be understood that the term “at least one” as used herein means that one or more of the items referred to following the term may be used in accordance with the invention. For example, if the term indicates that at least one sampling unit shall be used this may be under-stood as one sampling unit or more than one sampling units, i.e. two, three, four, five or any other number. Depending on the item the term refers to, the skilled person understands as to what upper limit the term may refer, if any.

The term “about” as used herein means that with respect to any number recited after said term an interval accuracy exists within in which a technical effect can be achieved.

Accordingly, about as referred to herein, preferably, refers to the precise numerical value or a range around said precise numerical value of +20%, preferably +15%, more preferably +10%, or even more preferably =5%.

Furthermore, the terms “first”, “second”, “third” and the like in the description and in the claims are used for distinguishing between similar elements and not necessarily for describing a sequential or chronological order.

The method of the present invention may consist of the aforementioned step or may comprise additional steps, such as steps for further evaluation of the assessment obtained in step (d), steps recommending therapeutic measures such as treatments, or the like. Moreover, it may comprise steps prior to step (a) such as steps relating to sample pre-treatments. However, preferably, it is envisaged that the above-mentioned method is an ex vivo method which does not require any steps being practiced on the human or animal body. Moreover, the method may be assisted by automation. Typically, the determination of the biomarkers may be supported by robotic equipment while the comparison and assessment may be supported by data processing equipment such as computers.

The term “assessing” as used herein refers to assessing whether a subject suffers from sepsis, is at risk of suffering from sepsis, exhibits a medical condition which deteriorates with respect to the overall health condition or with respect to sepsis or signs and symptoms accompanying sepsis and/or infection. Accordingly, assessing as used herein includes diagnosing sepsis, predicting the risk for developing sepsis, and/or predicting any deterioration of the health condition of the subject, in particular, with respect to signs and symptoms accompanying sepsis and/or infection.

In an embodiment, the term “assessment” refers to the diagnosis of sepsis. Thus, it is diagnosed whether a subject with suspected infection suffers from sepsis, or not.

In another embodiment, the assessment referred to in accordance with the present invention is the assessment of the risk of developing sepsis (and thus the prediction of the risk of developing sepsis). Moreover, it will be understood that if the risk of developing sepsis or risk of the deterioration of the health condition is predicted, typically, the prediction is made within a predictive window. More typically, said predictive window is about 8 h, about 10 h, about 12 h, about 16 h, about 20 h, about 24 h, about 48 h, in particular at least about 48 h, preferably, after the sample has been obtained. Further, risk of developing sepsis within 24 or 48 hours, preferably after the test sample has been obtained, may be predicted. In an embodiment of the prediction, the subject to be tested does not suffer from sepsis at the time at which the sample is obtained. Thus, the present invention allows for the early identification of patients at risk.

In an embodiment, the risk of developing sepsis within 24 hours is predicted.

In an alternative embodiment, the risk of developing sepsis within 48 hours is predicted. The period of 48 hours was assessed in the Examples section.

In yet another embodiment, the assessment is the prediction of the risk that the subject's (health) condition will deteriorate in the future, or not. The term “deterioration of the condition” of a subject who is suspected to suffer from an infection and/or who is suffering from an infection is well understood by the skilled person. The term typically relates to deterioration of the condition which may ultimately lead to further medication or other intervention.

Preferably, the condition of the subject deteriorates, if the subject's disease severity increases, if the subject's antibiotic therapy is intensified, if the subject is admitted to the ICU or to another unit for higher level of care, if the subject requires emergency surgery, if the subject dies in the hospital, if the subject dies within 30 days of admission, if the subject is re-hospitalized within 30 days of discharge, if the subject experiences organ dysfunction or failure, as measured e.g. with the SOFA score, and/or if the subject requires organ support.

The skilled person understands when the condition of a subject does not deteriorate. Typically, the condition of the subject does not deteriorate, if the subject does not have the outcomes mentioned in the previous paragraph.

In an embodiment, the condition of the subject deteriorates, if the subject has one or more of the following outcomes: if the subject admitted to the ICU, if the subject dies in the hospital, if the subject dies within 30 days of admission, and/or if the subject is re-hospitalized within 30 days of discharge.

In an embodiment, the prediction of the risk that the condition of the subject will deteriorate is the prediction of the risk that subject's antibiotic therapy is intensified.

In an embodiment, the prediction of the risk that the condition of the subject will deteriorate is the prediction of the risk of a subject to be admitted to ICU. Thus, it is assessed whether the subject is at risk of being admitted to the ICU, or not.

In another embodiment, the prediction of the risk that the condition of the subject will deteriorate is the prediction of the subject's risk of death in hospital. Thus, it is assessed whether the subject is at risk of death in hospital, or not.

In yet another embodiment, the prediction of the risk that the condition of the subject will deteriorate is the prediction of the subject's risk of death within 30 days of admission. Thus, it is assessed whether the subject is at risk of death within 30 days of admission to the hospital, or not.

In yet another embodiment, the prediction of the risk that the condition of the subject will deteriorate is the prediction of the subject's risk of re-hospitalization within 30 days of discharge. Thus, it is assessed whether the subject is at risk of re-hospitalization within 30 days of discharge, or not.

In yet another embodiment, the prediction of the risk that the condition of the subject will deteriorate is the prediction of the risk that the subject experiences organ dysfunction or failure. Organ dysfunction and failure can be e.g. assessed via the SOFA score. Accordingly, the present invention further is directed to the prediction of the risk that the SOFA score of the subject will increase, or not (after the test sample has been obtained). An increase of the SOFA score (such as by at least one, at least two, at least three, or at least four points etc.) is considered as a deterioration of the condition. In contrast, the condition typically does not deteriorate, if the SOFA score does not increase (provided that the subject does not have the highest SOFA score). The predictive window may be a predictive window as described above for the prediction of the risk to develop sepsis.

The sequential organ failure assessment (SOFA) is a validated score, combining clinical assessment and laboratory measures, that quantitatively describes organ dysfunction/failure. Dysfunction of respiration, coagulation, the liver, the cardiovascular system, the central nervous system and the kidney are scored individually, and are summed up to the SOFA score, which ranges from 0 to 24. Preferably, the SOFA score is determined as described in Vincent 1996 (Vincent et al. Intensive Care Med. 1996 July;22 (7): 707-10. doi: 10.1007/BF01709751. PMID: 8844239.).

In yet another embodiment, the prediction of the risk that the condition of the subject will deteriorate is the prediction of the risk that the subject requires organ support, such as the prediction of the risk that the subject requires vasoactive therapy, hemodynamic support (such as fluid therapy), oxygen supply (e.g. by ventilation or by extracorporeal membrane oxygenation), and/or renal replacement therapy. The predictive window may be a predictive window as described above for the prediction of the risk to develop sepsis, for example within 24 or 48 hours after the sample has been obtained.

In an embodiment, the term “assessment” refers to the diagnosis of sepsis. Thus, it is diagnosed whether a subject with suspected infection suffers from sepsis, or not. Preferably, the assessment refers to the early detection of sepsis.

As will be understood by those skilled in the art, the assessment made in accordance with the present invention, although preferred to be, may usually not be correct for 100% of the investigated subjects. The term, typically, requires that a statistically significant portion of subjects can be correctly assessed. Whether a portion is statistically significant can be determined without further ado by the person skilled in the art using various well known statistic evaluation tools, e.g., determination of confidence intervals, p-value determination, Student's t-test, Mann-Whitney test, etc., Details may be found in Dowdy and Wearden, Statistics for Research, John Wiley & Sons, New York 1983. Typically envisaged confidence intervals are at least 50%, at least 60%, at least 70%, at least 80%, at least 90%, at least 95%. The p-values are, typically, 0.2, 0.1, 0.05.

The term “subject” as used herein refers to an animal, preferably a mammal and, more typically to a human. The subject to be investigated by the method of the present invention shall be a subject having suspected infection. The term “suspected infection” as used herein means that the subject shall exhibit clinical parameters, signs and/or symptoms of infection. Thus, the subject according to the invention is, typically, a subject that suffers from an infection or is suspected to suffer from an infection. Typically, the subject is a subject presenting at the emergency department. Advantageously, the sample has been obtained at presentation. Preferably, the sample has been obtained at presentation at the emergency department. However, the sample may be also obtained at presentation at the primary care physician.

Typically, the subject to be tested shall be suspected to suffer from an infection. The term “infection” is well understood by the skilled person. As used herein, the term “infection” preferably refers to an invasion of the subject's body tissues by a disease-causing microorganism, its multiplication, and the reaction of subject's tissues to the microorganism. In an embodiment, the infection is a bacterial infection. Thus, the subject shall be suspected to suffer from bacterial infection.

The term “sample” as used herein refers to any sample that under physiological conditions comprises the first, second and/or third biomarkers referred to herein. More typically, the sample is a body fluid sample, e.g. a blood sample or sample derived therefrom, a urine sample, a saliva sample, an interstitial fluid sample, a lymphatic fluid sample or the like. Most typically, said sample is a blood sample or a sample derived therefrom. Accordingly, the sample may be a blood, serum or plasma sample. Blood samples include capillary, venous or arterial blood samples.

In an embodiment, the sample is an interstitial fluid sample.

The term “sepsis” is well-known in the art. As used herein, the term refers a life-threatening organ dysfunction caused by a dysregulated host response to infection. A definition for sepsis, for example, can be found in Singer et al. (Sepsis-3 The Third International Consensus Definitions for Sepsis and Septic Shock. JAMA 2016; 315:801-819) which herewith is incorporated by reference with respect to the entire disclosure content.

Preferably, the term “sepsis” refers to sepsis according to the Sepsis-3 definition as disclosed in Singer et al. (loc. cit.).

As set forth elsewhere herein, the present invention allows for the early identification of patients at risk. In an embodiment of the prediction as set forth herein, the subject to be tested thus does not suffer from sepsis at the time at which the sample is obtained. In particularly preferred embodiment, the subject to be tested does not suffer from septic shock, preferably, at the time at which the sample is obtained. The term “septic shock” is defined in Singer et al. (loc. cit.). Thus, a subject suffers from septic shock if the following criteria are met.
Sepsis, i.e. suspected/documented infection AND change in total SOFA score >2 points consequent to the infection
AND persisting hypotension requiring vasopressors to maintain MAP≥65 mm Hg and having a serum lactate level>2 mmol/L (18 mg/dL) despite adequate volume resuscitation

Further, it is envisaged that subject to be tested may or may not suffer from infection with SARS-COV-2.

The term “determining” as used herein refers to qualitative and quantitative determination of the biomarkers referred to in accordance with the present invention, i.e. the term encompasses the determination of the presence or absence or the determination of the absolute or relative amount of said biomarkers.

The term “amount” as used herein refers to the absolute amount of a compound referred to herein, the relative amount or concentration of the said compound as well as any value or parameter which correlates thereto or can be derived therefrom. Such values or parameters comprise intensity signal values from all specific physical or chemical properties obtained from the said compounds by direct measurements, e.g., intensity values in mass spectra or NMR spectra. Moreover, encompassed are all values or parameters which are obtained by indirect measurements specified elsewhere in this description, e.g., response levels determined from biological read out systems in response to the compounds or intensity signals obtained from specifically bound ligands. It is to be understood that values correlating to the aforementioned amounts or parameters can also be obtained by all standard mathematical operations.

Determining the amount in the method of the present invention may be carried out by any technique which allows for detecting the presence or absence or the amount of said second molecule upon its release from the first molecule. Suitable techniques depend on the molecular nature and the properties of the biomarkers and are discussed elsewhere herein in more detail.

Typically, the amount of a biomarker as referred to in accordance with the present invention can be determined by immunoassays using sandwich, competition, or other assay formats. Said assays will develop a signal which is indicative for the presence or absence or the amount of a biomarker. Further suitable methods comprise measuring a physical or chemical property specific for the biomarker such as its precise molecular mass or NMR spectrum. Said methods comprise, preferably, biosensors, optical devices coupled to immunoassays, biochips, analytical devices such as mass-spectrometers, NMR-analysers, surface plasmon resonance measurement equipment or chromatography devices. Further, methods include micro-plate ELISA-based methods, fully-automated or robotic immunoassays (available, e.g., from Roche). Suitable measurement methods according the present invention may also include precipitation (particularly immunoprecipitation), electrochemiluminescence (electro-generated chemiluminescence), RIA (radioimmunoassay), ELISA (enzyme-linked immunosorbent assay), electrochemiluminescence sandwich immunoassays (ECLIA), dissociation-enhanced lanthanide fluoro immuno assay (DELFIA), scintillation proximity assay (SPA), turbidimetry, nephelometry, latex-enhanced turbidimetry or nephelometry, or solid phase immune tests. Further methods known in the art such as gel electrophoresis, 2D gel electrophoresis, SDS polyacrylamid gel electrophoresis (SDS-PAGE) or Western Blotting. More typically, techniques particular envisaged for determining the biomarkers referred to herein are described in the accompanying Examples, below.

The biomarkers to be determined in accordance with the present invention are well-known in the art. Moreover, methods for the determination of the amount of the biomarkers are known. For example, the biomarkers can be measured as described in the Examples section (see Example 1).

Delta-like proteins are single-pass transmembrane proteins known for their role in Notch signaling as homologs of the Notch Delta ligand first described in Drosophila. The DLL1 (Delta-like ligand 1) polypeptide is a human homolog of the Notch Delta ligand and is a member of the delta/serrate/jagged family. It plays a role in mediating cell fate decisions during hematopoiesis. It may play a role in cell-to-cell communication. Synonyms of DLL1 are DELTA1, DL1, Delta or delta like canonical Notch ligand 1.

DLL1 (UniProtKB-000548 (DLL1_HUMAN)) binds the extracellular domain of Notch receptor. Upon interaction between a Notch receptor and DLL1, both the receptor and its ligand are cleaved from the surface, resulting in the generation of soluble extracellular part of DLL1 (sDLL1) which is released. The transmembrane domain and the intracellular domain remain linked to the cell. “DLL1” as used herein, preferably, refers to soluble DLL1, i.e. the released extracellular domain of DLL1.

The biomarker Mid-regional proadrenomedullin (MRproADM) is well known in the art. The biomarker has been proposed as a marker for sepsis (Christ-Crain, M., Morgenthaler, N. G., Struck, J. et al. Mid-regional pro-adrenomedullin as a prognostic marker in sepsis: an observational study. Crit Care 9, R816 (2005). https://doi.org/10.1186/cc3885). MRproADM is a fragment of with a length of 48 amino acids which is derived from the proADM molecule in a 1:1 ratio with Adrenomedullin (AM). Accordingly, the amount of MRproADM represents the amount and activity of Adrenomedullin. AM (Adrenomedullin) and PAMP (Proadrenomedullin N-terminal peptide) are potent hypotensive and vasodilatator agents. Numerous actions have been reported most related to the physiologic control of fluid and electrolyte homeostasis

The term “Growth-Differentiation Factor-15” or “GDF-15” relates to a polypeptide being a member of the transforming growth factor (TGF) cytokine superfamily. The terms polypeptide, peptide and protein are used interchangeable throughout this specification. GDF-15 was originally cloned as macrophage-inhibitory cytokine 1 and later also identified as placental transforming growth factor-15, placental bone morphogenetic protein, non-steroidal anti-inflammatory drug-activated gene 1, and prostate-derived factor (Bootcov loc cit; Hromas, 1997 Biochim Biophys Acta 1354:40-44; Lawton 1997, Gene 203:17-26; Yokoyama-Kobayashi 1997, J Biochem (Tokyo), 122:622-626; Paralkar 1998, J Biol Chem 273:13760-13767). Amino acid sequences for GDF-15 are disclosed in WO99/06445, WO00/70051, WO2005/113585, Bottner 1999, Gene 237:105-111, Bootcov loc. cit, Tan loc. cit., Baek 2001, Mol Pharmacol 59:901-908, Hromas loc cit, Paralkar loc cit, Morrish 1996, Placenta 17:431-441.

The term “soluble Flt-1” or “sFlt-1” (abbreviation for “Soluble fms-like tyrosine kinase-1”) as used herein, preferably, refers to polypeptide which is a soluble form of the VEGF receptor Flt1. It was identified in conditioned culture medium of human umbilical vein endothelial cells. The endogenous soluble Flt1 (sFlt-1) receptor is chromatographically and immunolog-ically similar to recombinant human sFlt-1 and binds [125]] VEGF with a comparable high affinity. Human sFlt-1 is shown to form a VEGF-stabilized complex with the extracellular domain of KDR/Flk-1 in vitro. Preferably, sFlt-1 refers to human sFlt-1 as described in Kendall 1996, Biochem Biophs Res Commun 226 (2): 324-328 (for amino acid sequences, see, e.g., also P17948, GI: 125361 for human and BAA24499.1, GI: 2809071 for mouse sFlt-1).

The marker Cystatin C is well known in the art. Cystatin C is encoded by the CST3 gene and is produced by all nucleated cells at a constant rate and the production rate in humans is remarkably constant over the entire lifetime. Elimination from the circulation is almost entirely via glomerular filtration. For this reason the serum concentration of cystatin C is independent from muscle mass and gender in the age range 1 to 50 years. Therefore cystatin

C in plasma and serum has been proposed as a more sensitive marker for GFR. The sequence of the human Cystatin C polypeptide can be assessed via Genbank (see e.g. accession number NP_000090.1). The biomarker can be determined by particle enhanced immunoturbidimetric assay. Human cystatin C agglutinates with latex particles coated with anti-cystatin C antibodies.

In the method according to the present invention, a third biomarker may be determined. In particular, step (b) of the method of the invention may further comprise determining the amount of sFlt1, Cystatin C or MR-proADM as a third biomarker.

Thus, the present invention concerns the determination of at least two biomarkers, i.e. the biomarker DLL1 and the biomarker GDF15, and optionally a third biomarker.

In an embodiment, the third biomarker is sFlt1. Thus, DLL1, GDF15 and sFLT1 are determined.

In an alternative embodiment, the third biomarker is Cystatin C (Cys). Thus, DLL1, GDF15 and CysC are determined.

In an alternative embodiment, the third biomarker is MR-proADM. Thus, DLL1, GDF15 and CysC are determined.

It is to be understood that that the invention is not limited to the above markers. Rather, the invention may encompass the determination of additional markers.

The term “reference” as used herein refers to an amount or value which allows for allocation of a subject into either the group of subjects suffering from a disease or condition or being at risk for developing it or the group of subjects which do not suffer from said disease or condition or which are not at risk for developing it. Such a reference can be a threshold amount which separates these groups from each other. Accordingly, the reference shall be an amount or score which allows for allocation of a subject into a group of subjects suffering from a disease or condition or being at risk for developing it, or not. For example, the reference shall be an amount or score which allows for allocation of a subject into a group of subjects being at risk of developing sepsis, or not being at risk of developing sequence (within a predictive window as set forth above, such as within about 48 hours).

A suitable threshold amount separating the two groups can be calculated without further ado by the statistical tests referred to herein elsewhere based on amounts of biomarkers from either a subject or group of subjects known to suffer from a disease or condition or being at risk for developing it or a subject or group of subjects known not to suffer from a disease or condition or being at risk for developing it. The reference amount applicable for an individual subject may vary depending on various physiological parameters such as age, gender, or subpopulation.

Typically, said references are references for each biomarker derived from at least one subject known to be at risk for developing sepsis, preferably wherein amounts for each of the biomarkers being essentially identical or similar to the corresponding references are indicative for a subject being at risk for developing sepsis, while amounts for each of the biomarkers being different from the corresponding references are indicative for a subject being not at risk for developing sepsis.

Also typically, said references are references for each biomarker derived from at least one subject known not to be at risk for developing sepsis, preferably wherein amounts for each of the biomarkers being essentially identical or similar to the corresponding references are indicative for a subject being not at risk for developing sepsis, while amounts for each of the biomarkers being different from the corresponding references are indicative for a subject being at risk for developing sepsis.

The term “at least one subject” refers to one subject or more than one subject, such as at least 10, 50, 100, 200, or 1000 subjects.

In an embodiment, amounts of the biomarkers larger than the references for said biomarkers are indicative for a subject being at risk (e.g. of developing sepsis, e.g. within a certain time period after the sample has been obtained). Further, amounts of the biomarkers lower than the references for said biomarkers are indicative for a subject not being at risk or not suffering from sepsis.

Reference amounts can, in principle, be calculated for a cohort of subjects based on the average or mean values for a given parameter such as biomarker amount by applying standard statistically methods. In particular, accuracy of a test such as a method aiming to diagnose an event, or not, is best described by its receiver-operating characteristics (ROC) (see especially Zweig 1993, Clin. Chem. 39:561-577). The ROC graph is a plot of all of the sensitivity/specificity pairs resulting from continuously varying the decision threshold over the entire range of data observed. The clinical performance of a diagnostic method depends on its accuracy, i.e. its ability to correctly allocate subjects to a certain prognosis or diagnosis. The ROC plot indicates the overlap between the two distributions by plotting the sensitivity versus 1-specificity for the complete range of thresholds suitable for making a distinction. On the y-axis is sensitivity, or the true-positive fraction, which is defined as the ratio of number of true-positive test results to the product of number of true-positive and number of false-negative test results. This has also been referred to as positivity in the presence of a disease or condition. It is calculated solely from the affected subgroup. On the x-axis is the false-positive fraction, or 1-specificity, which is defined as the ratio of number of false-positive results to the product of number of true-negative and number of false-positive results. It is an index of specificity and is calculated entirely from the unaffected subgroup. Because the true- and false-positive fractions are calculated entirely separately, by using the test results from two different subgroups, the ROC plot is independent of the prevalence of the event in the cohort. Each point on the ROC plot represents a sensitivity/-specificity pair corresponding to a particular decision threshold. A test with perfect discrimination (no overlap in the two distributions of results) has an ROC plot that passes through the upper left corner, where the true-positive fraction is 1.0, or 100% (perfect sensitivity), and the false-positive fraction is 0 (perfect specificity). The theoretical plot for a test with no discrimination (identical distributions of results for the two groups) is a 45° diagonal line from the lower left corner to the upper right corner. Most plots fall in between these two extremes. If the ROC plot falls completely below the 45° diagonal, this is easily remedied by reversing the criterion for “positivity” from “greater than” to “less than” or vice versa. Qualitatively, the closer the plot is to the upper left corner, the higher the overall accuracy of the test. Dependent on a desired confidence interval, a threshold can be derived from the ROC curve allowing for the diagnosis or prediction for a given event with a proper balance of sensitivity and specificity, respectively. Accordingly, the reference to be used for the aforementioned method of the present invention, i.e. a threshold which allows to discriminate between subjects being at risk and not being at risk (e.g. of developing sepsis) can be generated, usually, by establishing a ROC for said cohort as described above and deriving a threshold amount therefrom. Dependent on a desired sensitivity and specificity for a diagnostic method, the ROC plot allows deriving suitable thresholds. It will be understood that an optimal sensitivity is desired for excluding a subject for being at increased risk or for suffering from a disease (i.e. a rule out) whereas an optimal specificity is envisaged for a subject to be assessed as being at an increased risk or to suffer from the disease (i.e. a rule in).

Step c) of the method of the present invention comprises comparing the amounts of the biomarkers (i.e. the first biomarker, the second biomarker and optionally the third biomarker) to references for said biomarkers and/or calculating a score for assessing the subject with suspected infection based on the amounts of the biomarkers.

Thus, the amount of the first biomarker, the second biomarker and optionally the third biomarker, respectively, may be compared to a reference for the first biomarker, a reference for the second biomarker and optionally a reference for the third biomarker.

Alternatively, a score may be calculated based on the amounts the biomarkers, i.e. based on the amounts of the first biomarker, the second biomarker and, optionally the third biomarker. Said score shall allow for assessing the subject with suspected infection, such as for predicting the risk of developing sepsis. Optionally, said score may be compared to a suitable reference score.

The term “comparing” as used herein encompasses comparing the determined amount for a biomarker as referred to herein to a reference. It is to be understood that comparing as used herein refers to any kind of comparison made between the value for the amount with the reference. However, it is to be understood that, preferably, identical types of values are compared with each other, e.g., if an absolute amount is determined and to be compared in the method of the invention, the reference shall also be an absolute amount, if a relative amount is determined and to be compared in the method of the invention, the reference shall also be a relative amount, etc., Alternatively, the term “comparing” as used herein encompasses comparing a calculated score with a suitable reference core. The comparison may be carried out manually or computer assisted. The value of the amount and the reference can be, e.g., compared to each other and the said comparison can be automatically carried out by a computer program executing an algorithm for the comparison. The computer program carrying out the said evaluation will provide the desired assessment in a suitable output format.

As set forth above, it is also envisaged to calculate a score (in particular a single score) based on the amounts of the first and second biomarker, or the first, second or third biomarker, i.e. a single score, and to compare this score to a reference score. Preferably, the score is based on the amounts of the first and second biomarker in the sample from the test subject, and, if the amount of the third biomarker is determined, on the amounts of first, second and third biomarker in the sample from the test subject.

The calculated score combines information on the amounts of the at least two biomarkers (e.g. of the two or three biomarkers). Moreover, in the score, the biomarkers are, preferably, weighted in accordance with their contribution to the establishment of the assessment, such as the differentiation. Thus, the values for the individual markers are weighted and the weighted values are used for calculating the score. Suitable coefficients (weights) can be determined by the skilled person without further ado. A score can also be calculated from a decision tree or a set (ensemble) of decision trees that has been trained on at least two biomarkers. Based on the combination of biomarkers applied in the method of the invention, the weight of an individual biomarker as well as the structure of decision trees may be different.

The score can be regarded as a classifier parameter for assessing a subject as set forth herein. In particular, it enables the person who provides the assessment based on a single score. The reference score is preferably a value, in particular a cut-off value which allows for assessing a subject with suspected infection as set forth herein. Preferably, the reference is a single value. Thus, the person does not have to interpret the entire information on the amounts of the individual biomarkers. Using a scoring system as described herein, advantageously, values of different dimensions or units for the biomarkers may be used since the values will be mathematically transformed into the score. Accordingly, e.g. values for absolute concentrations may be combined in a score with peak area ratios. The reference score to be applied may be elected based on the desired sensitivity or the desired specificity. How to elect a suitable reference score is well known in the art.

Advantageously, it has been found in the studies underlying the present invention that a combination of a first biomarker with a second and, preferably, a third biomarker allows for a reliable and early assessment of patients exhibiting signs and symptoms of infection. In the studies, patients presenting at emergency departments being medical (non-surgical) emergencies were investigated. To this end, patients were subdivided into those that are suffering from sepsis with a high probability and those suspected to suffer from infection without sepsis. The patients with suspected infection were further subdivided into those whose overall condition deteriorated and into those whose overall condition did not deteriorate. Deterioration was defined as: escalation of care (i.e. admission to the ICU), death in hospital, death within 30 days of admission, or re-hospitalization within 30 days of discharge. The amount of various biomarkers has been determined and the biomarkers were analyzed and mathematically combined via logic regression analysis. The area under the receiver operating characteristic (AUC) was used to evaluate biomarker performance. The AUC values are the mathematical integer of a function f(x) within the interval [a] [b]. AUC was also investigated for biomarker pairs and triplets. Biomarker combinations which together showed improved AUC over the best single biomarker AUC were identified. The results are described in the accompanying Examples, below.

In particular, if these patients are presenting in, e.g., emergency units, an early assessment of the risk for developing severe complications such as sepsis, SIRS or general deterioration of their overall health condition is decisive to start therapeutic measures including drug administration, physical or other therapeutic interventions and/or hospitalization. These therapeutic measures, in particular, may include, e.g., rapid administration of broad spectrum antibiotics, fluid resuscitation, vasoactive drug therapy, mechanical ventilation, other organ support (e.g., continuous hemofiltration, extracorporeal membrane oxygenation). Also encompassed as therapeutic measures is triage to higher level of care (e.g. intensive care unit, intermediate care unit). If there is no risk for severe complication, patients could be discharged home and managed in the outpatient setting or admitted to the hospital at a low level of care (e.g. general ward). Thanks to the present invention, life-threatening developments can be prevented since patients can be assessed by biomarker determination at an early stage. The biomarker pairs and triplets identified in the studies underling the present invention are a reliable basis for medical decisions and the assessment can be performed in a time- and cost-effective manner.

Thus, the methods of the present invention may further comprise recommending or initiating a suitable therapeutic measure. Typically, said suitable therapeutic measure is selected from the medical guidelines or recommendations for management of sepsis such as International Guidelines for Management of Sepsis and Septic Shock (Intensive Care Med, 2017). For example, the therapeutic measure may be treatment of sepsis or further diagnostic investigation or other aspects of care deemed necessary by the practitioners.

In an embodiment, the therapeutic measure to be recommended or initiated if a patient has been assessed to be at risk is selected from
administration of empiric broad spectrum therapy with at least one or more broad spectrum antibiotics, such as a cephalosporine, a beta-lactam/beta-lactamase inhibitor (e.g. piperacillin), or a carbapenem, typically depending on the organisms that are considered likely pathogens and antibiotic susceptibilities
fluid resuscitation
administration of one or more vasopressors, such as administration of norepinephrine, and
administration of one or more corticosteroids, such as administration of hydrocortisone

The definitions given herein above, apply mutatis mutandis to the following.

The present invention also relates to a computer-implemented method for assessing a subject with suspected infection comprising the steps of:
(a) receiving a value for the amount of a first biomarker in a sample of the subject, said first biomarker being DLL1;
(b) receiving a value for the amount of a second biomarker in a sample of the subject, said second biomarker being GDF15;
(c) comparing the values for the amounts of the biomarkers to references for said biomarkers and/or calculating a score for assessing the subject with suspected infection based on the amounts of the biomarkers; and
(d) assessing said subject based on the comparison and/or the calculation made in step (c).

The term “computer-implemented” as used herein means that the method is carried out in an automated fashion on a data processing unit which is, typically, comprised in a computer or similar data processing device. The data processing unit shall receive values for the amount of the biomarkers. Such values can be the amounts, relative amounts or any other calculated value reflecting the amount as described elsewhere herein in detail. Accordingly, it is to be understood that the aforementioned method does not require the determination of amounts for the biomarkers but rather uses values for already predetermined amounts.

Typically, in step (b) of said method, the method may comprise receiving a value for the amount of sFlt1, Cystatin C or MR-proADM as a third biomarker.

The present invention also, in principle, contemplates a computer program, computer program product or computer readable storage medium having tangibly embedded said computer program, wherein the computer program comprises instructions which, when run on a data processing device or computer, carry out the method of the present invention as specified above. Specifically, the present disclosure further encompasses:
a computer or computer network comprising at least one processor, wherein the processor is adapted to perform the method according to one of the embodiments described in this description,
a computer loadable data structure that is adapted to perform the method according to one of the embodiments described in this description while the data structure is being executed on a computer,
a computer script, wherein the computer program is adapted to perform the method according to one of the embodiments described in this description while the program is being executed on a computer,
a computer program comprising program means for performing the method according to one of the embodiments described in this description while the computer program is being executed on a computer or on a computer network,
a computer program comprising program means according to the preceding embodiment, wherein the program means are stored on a storage medium readable to a computer,
a storage medium, wherein a data structure is stored on the storage medium and wherein the data structure is adapted to perform the method according to one of the embodiments described in this description after having been loaded into a main and/or working storage of a computer or of a computer network,
a computer program product having program code means, wherein the program code means can be stored or are stored on a storage medium, for performing the method according to one of the embodiments described in this description, if the program code means are executed on a computer or on a computer network,
a data stream signal, typically encrypted, comprising a data for parameters as defined herein elsewhere, and
a data stream signal, typically encrypted, comprising the assessment provided by the methods of the present invention.

The present invention relates to a device for assessing a subject with suspected infection comprising:
(a) a measuring unit for determining the amount of a first biomarker being DLL1 and a second biomarker being GDF15 in a sample of the subject, said measuring unit comprising a detection system for the first biomarker and the second biomarker; and
(b) an evaluation unit operably linked to the measuring unit comprising a database with stored references for the first biomarker and the second biomarker, preferably, as specified above and a data processor comprising instructions for carrying out a comparison of the amount of the first biomarker and the second biomarker to references and/or for carrying out a calculation of a score for assessing the subject with suspected infection based on the amounts of the biomarkers, preferably, as specified above and for assessing said subject based on the comparison, said evaluation unit being capable of automatically receiving values for the amounts of the biomarkers from the measuring unit.

The term “device” as used herein relates to a system comprising the aforementioned units operatively linked to each other as to allow the determination of the amounts of biomarkers and evaluation thereof according to the method of the invention such that an assessment can be provided.

The analyzing unit, typically, comprises at least one reaction zone having a biomarker detection agent for the first and second biomarker and, preferably also the third biomarker, in immobilized form on a solid support or carrier which is to be contacted to the sample. Moreover, in the reaction zone, it is possible to apply conditions which allow for the specific binding of the detection agent(s) to the biomarkers comprised in the sample.

The reaction zone may either allow directly for sample application or it may be connected to a loading zone where the sample is applied. In the latter case, the sample can be actively or passively transported via the connection between the loading zone and the reaction zone to the reaction zone. Moreover, the reaction zone shall be also connected to a detector. The connection shall be such that the detector can detect the binding of the biomarkers to their detection agents. Suitable connections depend on the techniques used for measuring the presence or amount of the biomarkers. For example, for optical detection, transmission of light may be required between the detector and the reaction zone while for electrochemical determination a fluidal connection may be required, e.g., between the reaction zone and an electrode.

The detector shall be adapted to detect determination of the amount of the biomarkers. The determined amount can be subsequently transmitted to the evaluation unit. Said evaluation unit comprises a data processing element, such as a computer, with an implemented algorithm for determining the amount present in the sample.

The processing unit as referred to in accordance with the method of the present invention, typically, comprises a Central Processing Unit (CPU) and/or one or more Graphics Processing Units (GPUs) and/or one or more Application Specific Integrated Circuits (ASICs) and/or one or more Tensor Processing Units (TPUs) and/or one or more field-programmable gate arrays (FPGAs) or the like. A data processing element may be a general purpose computer or a portable computing device, for example. It should also be understood that multiple computing devices may be used together, such as over a network or other methods of transferring data, for performing one or more steps of the methods disclosed herein. Exemplary computing devices include desktop computers, laptop computers, personal data assistants (“PDA”), cellular devices, smart or mobile devices, tablet computers, servers, and the like. In general, a data processing element comprises a processor capable of executing a plurality of instructions (such as a program of software).

The evaluation unit, typically comprises or has access to a memory. A memory is a computer readable medium and may comprise a single storage device or multiple storage devices, located either locally with the computing device or accessible to the computing device across a network, for example. Computer-readable media may be any available media that can be accessed by the computing device and includes both volatile and non-volatile media. Further, computer readable-media may be one or both of removable and non-removable media. By way of example, and not limitation, computer-readable media may comprise computer storage media. Exemplary computer storage media includes, but is not limited to, RAM, ROM, EEPROM, flash memory or any other memory technology, CD-ROM, Digital Versatile Disk (DVD) or other optical disk storage, magnetic cassettes, magnetic tape, magnetic disk storage or other magnetic storage devices, or any other medium which can be used for storing a plurality of instructions capable of being accessed by the computing device and executed by the processor of the computing device.

According to embodiments of the instant disclosure, software may include instructions which, when executed by a processor of the computing device, may perform one or more steps of the methods disclosed herein. Some of the instructions may be adapted to produce signals that control operation of other machines and thus may operate through those control signals to transform materials far removed from the computer itself. These descriptions and representations are the means used by those skilled in the art of data processing, for example, to most effectively convey the substance of their work to others skilled in the art.

The plurality of instructions may also comprise an algorithm which is generally conceived to be a self-consistent sequence of steps leading to a desired result. These steps are those requiring physical manipulations of physical quantities. Usually, though not necessarily, these quantities take the form of electrical or magnetic pulses or signals capable of being stored, transferred, transformed, combined, compared, and otherwise manipulated. It proves convenient at times, principally for reasons of common usage, to refer to these signals as values, characters, display data, numbers, or the like as a reference to the physical items or manifestations in which such signals are embodied or expressed. It should be borne in mind, however, that all of these and similar terms are to be associated with the appropriate physical quantities and are merely used here as convenient labels applied to these quantities.

The evaluation unit may also comprise or has access to an output device. Exemplary output devices include fax machines, displays, printers, and files, for example. According to some embodiments of the present disclosure, a computing device may perform one or more steps of a method disclosed herein, and thereafter provide an output, via an output device, relating to a result, indication, ratio or other factor of the method.

Typically, said measuring unit determines and comprises a detection system for a third biomarker and wherein said database comprises stored a reference for a third biomarker, said third biomarker being sFlt1, Cystatin C or MR-proADM.

More typically, said detection system comprises at least one detection agent being capable of specifically detecting each of the biomarkers.

The present invention further contemplates a device for assessing a subject with suspected infection comprising an evaluation unit comprising a database with stored references for a first biomarker being DLL1 and a second biomarker being GDF15 and a data processor comprising instructions for carrying out a comparison of the amount of the first biomarker and the second biomarker to references, preferably, as specified above and for assessing said subject based on the comparison, said evaluation unit being capable of receiving values for the amounts of the biomarkers determined in a sample of the subject.

Typically, said database comprises a stored reference for a third biomarker, said third biomarker being sFlt1, Cystatin C or MR-proADM.

The present invention, in principle, also relates to the use of a first biomarker being DLL1 and a second biomarker being GDF15, or a detection agent specifically binding to said first biomarker and a detection agent specifically binding to said second biomarker for assessing a subject with suspected infection.

The term “detection agent” as used herein tyically refers to any agent which specifically binds to a biomarker, i.e. an agent which does not cross-react with other components present in the sample. Typically, a detection agent specifically binding a biomarker as referred to herein may be an antibody, an antibody fragment or derivative, an aptamer, a ligand for the biomarker, a receptor for the biomarker, an enzyme known to bind and/or convert the biomarker, or a small molecule known to specifically bind to the biomarker. For example, antibodies as referred to herein as detection agents include both polyclonal and monoclonal antibodies, as well as fragments thereof, such as Fv, Fab and F(ab)2 fragments that are capable of binding antigen or hapten. The present invention also includes single chain antibodies and humanized hybrid antibodies wherein amino acid sequences of a non-human donor antibody exhibiting a desired antigen-specificity are combined with sequences of a human acceptor antibody. The donor sequences will usually include at least the antigen-binding amino acid residues of the donor but may comprise other structurally and/or functionally relevant amino acid residues of the donor antibody as well. Such hybrids can be prepared by several methods well known in the art. Aptamer detection agents, e.g., may be nucleic acid or peptide aptamers. Methods to prepare such aptamers are well-known in the art. For example, random mutations can be introduced into the nucleic acids or peptides being the basis for aptamers. These derivatives can then be tested for binding according to screening procedures known in the art, e.g. phage display. Specific binding of a detection agent means that it should not bind substantially to, i.e. cross-react with, another peptide, polypeptide or substance present in the sample to be analyzed. Preferably, the specifically bound biomarker should be bound with at least 3 times higher, more preferably at least 10 times higher and even more preferably at least 50 times higher affinity than any other components of the sample. Non-specific binding may be tolerable, if it can still be distinguished and measured unequivocally, e.g. according to its size on a Western Blot, or by its relatively higher abundance in the sample.

The detection agent may be fused or linked permanently or reversibly to a detectable label. Suitable labels are well known to the skilled artisan. Suitable detectable labels are any labels detectable by an appropriate detection method. Typical labels include gold particles, latex beads, acridan ester, luminol, ruthenium, enzymatically active labels, radioactive labels, magnetic labels (“e.g. magnetic beads”, including paramagnetic and superparamagnetic labels), and fluorescent labels. Enzymatically active labels include e.g. horseradish peroxidase, alkaline phosphatase, beta-Galactosidase, Luciferase, and derivatives thereof. Suitable substrates for detection include di-amino-benzidine (DAB), 3,3′-5,5′ -tetramethylbenzidine, NBT-BCIP (4-nitro blue tetrazolium chloride and 5-bromo-4-chloro-3-indolyl-phosphate, available as ready-made stock solution from Roche Diagnostics), CDP-StarTM (Amersham Biosciences), ECFTM (Amersham Biosciences). A suitable enzyme-substrate combination may result in a colored reaction product, fluorescence or chemoluminescence, which can be measured according to methods known in the art (e.g. using a light-sensitive film or a suitable camera system). As for measuring the enyzmatic reaction, the criteria given above apply analogously. Typical fluorescent labels include fluorescent proteins (such as GFP and its derivatives), Cy3, Cy5, Texas Red, Fluorescein, and the Alexa dyes (e.g. Alexa 568). Further fluorescent labels are available e.g. from Molecular Probes (Oregon). Also the use of quantum dots as fluorescent labels is contemplated. Typical radioactive labels include 35S, 1251, 32P, 33P and the like. A radioactive label can be detected by any method known and appropriate, e.g. a light-sensitive film or a phosphor imager. Suitable labels may also be or comprise tags, such as biotin, digoxygenin, His-Tag, Glutathion-S-Transferase, FLAG, GFP, myc-tag, influenza A virus haemagglutinin (HA), maltose binding protein, and the like.

The determination of a biomarker as set forth herein may comprise mass spectrometry (MS) which is carried out after the separation step (e.g. by LC or HPLC). Mass spectrometry as used herein encompasses all techniques which allow for the determination of the molecular weight (i.e. the mass) or a mass variable corresponding to a compound, i.e. a biomarker, to be determined in accordance with the present invention. Preferably, mass spectrometry as used herein relates to GC-MS, LC-MS, direct infusion mass spectrometry, FT-ICR-MS, CE-MS, HPLC-MS, quadrupole mass spectrometry, any sequentially coupled mass spectrometry such as MS-MS or MS-MS-MS, ICP-MS, Py-MS, TOF or any combined approaches using the aforementioned techniques. How to apply these techniques is well known to the person skilled in the art. Moreover, suitable devices are commercially available. More preferably, mass spectrometry as used herein relates to LC-MS and/or HPLC-MS, i.e. to mass spectrometry being operatively linked to a prior liquid chromatography separation step. Preferably, the mass spectrometry is tandem mass spectrometry (also known as MS/MS). Tandem mass spectrometry, also known as MS/MS involves two or more mass spectrometry step, with a fragmentation occurring in between the stages. In tandem mass spectrometry two mass spectrometers in a series connected by a collision cell. The mass spectrometers are coupled to the chromatographic device. The sample that has been separated by a chromatography is sorted and weighed in the first mass spectrometer, then fragmented by an inert gas in the collision cell, and a piece or pieces sorted and weighed in the second mass spectrometer. The fragments are sorted and weighed in the second mass spectrometer. Identification by MS/MS is more accurate.

In an embodiment, mass spectrometry as used herein encompasses quadrupole MS. Most preferably, said quadrupole MS is carried out as follows: a) selection of a mass/charge quotient (m/z) of an ion created by ionisation in a first analytical quadrupole of the mass spectrometer, b) frag-mentation of the ion selected in step a) by applying an acceleration voltage in an additional subsequent quadrupole which is filled with a collision gas and acts as a collision chamber, c) selection of a mass/charge quotient of an ion created by the fragmentation process in step b) in an additional subsequent quadrupole, whereby steps a) to c) of the method are carried out at least once and analysis of the mass/charge quotient of all the ions present in the mixture of substances as a result of the ionisation process, whereby the quadrupole is filled with collision gas but no acceleration voltage is applied during the analysis. Details on said most preferred mass spectrometry to be used in accordance with the present invention can be found in WO2003/073464.

More preferably, said mass spectrometry is liquid chromatography (LC) MS such high performance liquid chromatography (HPLC) MS, in particular HPLC-MS/MS. Liquid chromatography as used herein refers to all techniques which allow for separation of compounds (i.e. metabolites) in liquid or supercritical phase.

For mass spectrometry, the analytes in the sample are ionized in order to generate charged molecules or molecule fragments. Afterwards, the mass-to-charge of the ionized analyte, in particular of the ionized biomarkers, or fragments thereof is measured. Prior to the ionization, the sample may be subjected to cleavage with a protease, e.g. with trypsin. The protease cleaves the protein biomarkers into smaller fragments.

Thus, the mass spectrometry step preferably comprises an ionization step in which the biomarkers to be determined are ionized. Of course, other compounds present in the sample/elulate are ionizied as well. Ionization of the biomarkers can be carried out by any method deemed appropriate, in particular by electron impact ionization, fast atom bombardment, electrospray ionization (ESI), atmospheric pressure chemical ionization (APCI), matrix assisted laser desorption ionization (MALDI).

In a preferred embodiment, the ionization step (for mass spectrometry) is carried out by electrospray ionization (ESI). Accordingly, the mass spectrometry is preferably ESI-MS (or if tandem MS is carried out: ESI-MS/MS). Electrospray is a soft ionization method which results in the formation of ions without breaking any chemical bonds.

More typically, a third biomarker or an detection agent specifically binding to said third biomarker is used in addition, said third biomarker being sFlt1, Cystatin C or MR-proADM.

The present invention also relates to a kit for assessing a subject with suspected infection comprising a detection agent specifically binding to a first biomarker being DLL1 and a detection agent specifically binding to a second biomarker being GDF15.

The term “kit” as used herein refers to a collection of the aforementioned components, typically, provided in separate or within a single container. The container also typically comprises instructions for carrying out the method of the present invention. These instructions may be in the form of a manual or may be provided by a computer program code which is capable of carrying out or supports the determination of the biomarkers referred to in the methods of the present invention when implemented on a computer or a data processing device. The computer program code may be provided on a data storage medium or device such as an optical storage medium (e.g., a Compact Disc) or directly on a computer or data processing device or may be provided in a download format such as a link to an accessible server or cloud. Moreover, the kit may, usually, comprise standards for reference amounts of biomarkers for calibration purposes as described elsewhere herein in detail. The kit according to the present invention may also comprise further components which are necessary for carrying out the method of the invention such as solvents, buffers, washing solutions and/or reagents required for detection of the released second molecule. Further, it may comprise the device of the invention either in parts or in its entirety.

More typically, said kit further comprises a detection agent specifically binding a third biomarker, said third biomarker being sFlt1, Cystatin C or MR-proADM.

The definitions and explanations given above apply mutatis mutandis to the following.

The determination of the amounts of the first, second and optionally the third biomarker as referred to herein in connection with the method of assessing a subject with suspected infection would also allow for monitoring a subject.

The present invention thus relates to a method for monitoring a subject, comprising:
(a) determining the amount of DLL1, the amount of GDF-15, and optionally the amount of the third biomarker as referred to herein (i.e. sFlt1, Cystatin C or MR-proADM) in first sample of said subject and, optionally, calculating a first score based on the determined amounts,
(b) determining the amount of DLL1, the amount of GDF-15, and optionally the amount of the third biomarker in a second sample of said subject and, optionally, calculating a second score based on the determined amounts; and
(c) comparing
   c1) the first score to the second score, or
   c2) the amounts of DLL1, of GDF-15 and optionally of the third biomarker in the second sample to the amounts of DLL1, of GDF-15 and optionally of the third biomarker in the first sample, whereby the subject is monitored.

Thus, the above method comprises two alternative embodiments.

According to embodiment c1), the first score is compared to the second score. This embodiment requires the calculation of a score in step (a) and step (b).

According to embodiment c2), the amounts of the biomarkers in the second sample are compared to the amounts of the biomarkers in the first sample. This embodiment does not require the calculation of a score in step (a) and step (b).

The term “subject” been defined above. The definition applies accordingly. Preferably, the subject is a subject with suspected infection, or a subjection suffering from an infection. Also preferably, the subject is a subject presenting at the emergency department.

In an embodiment, the term “monitoring a subject” refers to determining whether a patient is treated successfully by a therapeutic measure (which has been defined elsewhere herein). Thus, the term “monitoring a subject” preferably, relates to assessing whether a subject responds to a therapeutic measure as referred to herein, or not. Preferably, a subject responds to a therapeutic measure, if said therapy improves the condition of the subject (with respect to the infection). Preferably, a subject does not respond to said therapy, if said therapy does not improve the condition of the subject (with respect to the infection). The therapeutic measure, typically, has been initiated after obtaining the first sample, but before obtaining the second sample. However, it may have been also initiated before obtaining the first sample.

Also preferably, the term “monitoring a subject” relates to assessing whether the condition of the subject ameliorates or deteriorates. Monitoring may be used for active patient management including deciding on hospitalization, intensive care measures and/or additional qualitative monitoring as well as quantitative monitoring measures, i.e. monitoring frequency.

The term “sample” has been described elsewhere herein. For example, the sample may be a blood, serum or plasma sample.

The amounts of the biomarkers shall be determined in a first sample and a second sample. In an embodiment, the first sample has been obtained at presentation (e.g. at the emergency department). The “second sample” is particularly understood as a sample which is obtained in order to reflect a change of the amounts of the biomarker or the score in the second sample to the amount of the biomarkers or to the first score (i.e. the score in the first sample). Thus, the second sample, preferably, shall have been obtained after the first sample. It is to be understood that the second sample has been obtained not too early after the first sample in order to observe a sufficiently significant change of the score to allow for monitoring the patient. Therefore, the second sample has been, preferably, obtained at least 4 hours, or, more preferably, at least eight hours, or most preferably, at least 20 hours after the first sample has been obtained. In an embodiment, the second sample has been obtained 8 to 30 hours after the first sample, such as 12 to 26 hours.

Preferably, a decrease of the second score (or of the amounts of the biomarkers in the second sample) as compared to the first score (or to the amounts of the biomarkers in the first sample) shall be indicative for a subject who responds to a therapeutic measure or whose condition improves. In contrast, an increase of the second score as compared to the first score shall be indicative for a subject who does not respond to a therapeutic measure or whose condition did not improve. By carrying out the aforementioned method, decisions can be made whether a therapeutic measure in said subject shall be continued, stopped or amended.

Preferably, a subject responds to a therapeutic measure, if said therapy improves the condition of the subject. Preferably, a subject does not respond to said therapy, if said therapy does not improve the condition of the subject. In this case, the therapy may put the subject at risk of adverse side effects without any significant benefit to said subject (thereby generating useless health care costs).

Preferably, a decrease and, more preferably, a significant decrease, and, most preferably, a statistically significant decrease of the second score as compared to the first score is indicative for a subject who responds to a therapeutic measure.

A significant decrease, preferably, is a decrease of a size which is considered to be significant for monitoring a subject. Particularly said decrease is considered statistically significant. The terms “significant” and “statistically significant” are known by the person skilled in the art.

Thus, whether a decrease is significant or statistically significant can be determined without further ado by the person skilled in the art using various well known statistic evaluation tools. Preferred significant decreases of the score which are indicative for a subject who responds to a therapeutic measure are given herein below

A decrease, preferably, of at least 5%, of at least 10%, more preferably of at least 20%, and, even more preferably, of at least 30%, and most preferably of at least 40% is considered to be significant and, thus, to be indicative for a subject who responds to a therapeutic measure or whose conditions improved.

The present invention also relates to the in vitro use of i) a first biomarker being DLL1 and a second biomarker being GDF15, or ii) a detection agent specifically binding to said first biomarker and an detection agent specifically binding to said second biomarker for monitoring a subject. Preferably, the biomarkers or detection agents are used in a first and second sample as described above.

In an embodiment, a third biomarker or a detection agent specifically binding to said third biomarker is used in addition, said third biomarker being sFlt1, Cystatin C or MR-proADM.

It is to be understood that the definitions and explanations of the terms made above apply accordingly for all embodiments described in this specification and the accompanying claims.

The following embodiments are particular embodiments envisaged according to the present invention:
1. A method for assessing a subject with suspected infection comprising the steps of:
   (a) determining the amount of a first biomarker in a sample of the subject, said first biomarker being DLL1;
   (b) determining the amount of a second biomarker in a sample of the subject, said second biomarker being GDF15;
   (c) comparing the amounts of the biomarkers to references for said biomarkers and/or calculating a score for assessing the subject with suspected infection based on the amounts of the biomarkers; and
   (d) assessing said subject based on the comparison and/or the calculation made in step (c).
2. The method of embodiment 1, wherein step (b) further comprises determining the amount of MR-proADM, sFlt1 and/or Cystatin C.
3. The method of embodiment 1 or 2, wherein the subject is a subject presenting at the emergency department.
4. The method of any one of embodiments 1 to 3, wherein the assessment is the assessment of the risk of developing sepsis and/or the assessment of the risk that the subject's condition will deteriorate.
5. The method of any one of embodiments 1 to 4, wherein said references are references for each biomarker derived from at least one subject known to be at risk for developing sepsis, preferably wherein amounts for each of the biomarkers being essentially identical or similar to the corresponding references are indicative for a subject being at risk for developing sepsis while amounts for each of the biomarkers being different from the corresponding references are indicative for a subject being not at risk for developing sepsis.
6. The method of any one of embodiments 1 to 4, wherein said references are references for each biomarker derived from at least one subject known not to be at risk for developing sepsis, preferably wherein amounts for each of the biomarkers being essentially identical or similar to the corresponding references are indicative for a subject being not at risk for developing sepsis while amounts for each of the biomarkers being different from the corresponding references are indicative for a subject being at risk for developing sepsis.
7. The method of any one of embodiments 1 to 6, wherein said subject suffers from an infection or is suspected to suffer from an infection.
8. The method of any one of embodiments 1 to 7, wherein said sample is a blood sample or a sample derived therefrom.
9. The method of any one of embodiments 1 to 8, wherein said subject is a human.
10. A computer-implemented method for assessing a subject with suspected infection comprising the steps of:
   (a) receiving a value for the amount of a first biomarker in a sample of the subject, said first biomarker being DLL1;
   (b) receiving a value for the amount of a second biomarker in a sample of the subject, said second biomarker being GDF15;
   (c) comparing the values for the amounts of the biomarkers to references for said biomarkers and/or calculating a score for assessing the subject with suspected infection based on the amounts of the biomarkers; and
   (d) assessing said subject based on the comparison and/or the calculation made in step (c).
11. The method of embodiment 10, wherein in step (b) the method further comprises receiving a value for the amount of sFlt1, Cystatin C or MR-proADM as a third biomarker.
12. A device for assessing a subject with suspected infection comprising:
   (a) a measuring unit for determining the amount of a first biomarker being DLL1 and a second biomarker being GDF15 in a sample of the subject, said measuring unit comprising a detection system for the first biomarker and the second biomarker; and
   (b) an evaluation unit operably linked to the measuring unit comprising a database with stored references for the first biomarker and the second biomarker, preferably, as specified in any one of embodiments 1 to 9 and a data processor comprising instructions for carrying out a comparison of the amount of the first biomarker and the second biomarker to references and/or for carrying out a calculation of a score for assessing the subject with suspected infection based on the amounts of the biomarkers, preferably, as specified in any one of embodiments 1 to 9 and for assessing said subject based on the comparison, said evaluation unit being capable of automatically receiving values for the amounts of the biomarkers from the measuring unit.
13. The device of embodiment 12, wherein said measuring unit determines and comprises a detection system for a third biomarker and wherein said database comprises stored a reference for a third biomarker, said third biomarker being sFlt1, Cystatin C or MR-proADM.
14. The device of embodiment 12 or 13, wherein said detection system comprises at least one detection agent being capable of specifically detecting each of the biomarkers.
15. A device for assessing a subject with suspected infection comprising an evaluation unit comprising a database with stored references for a first biomarker being DLL1 and a second biomarker is being GDF15 and a data processor comprising instructions for carrying out a comparison of the amount of the first biomarker and the second biomarker to references, preferably, as specified in any one of embodiments 1 to 11 and for assessing said subject based on the comparison, said evaluation unit being capable of receiving values for the amounts of the biomarkers determined in a sample of the subject.
16. The device of embodiment 15, wherein said database comprises a stored reference for a third biomarker, said third biomarker being sFlt1, Cystatin C or MR-proADM.
17. Use of a i) first biomarker being DLL1 and a second biomarker being GDF15, or ii) a detection agent specifically binding to said first biomarker and an detection agent specifically binding to said second biomarker for assessing a subject with suspected infection.
18. The use of embodiment 17, wherein a third biomarker or an detection agent specifically binding to said third biomarker is used in addition, said third biomarker being sFlt1, Cystatin C or MR-proADM.
19. A kit for assessing a subject with suspected infection comprising an detection agent specifically binding to a first biomarker being DLL1 and an detection agent specifically binding to a second biomarker being GDF15.
20. The kit of embodiment 19, wherein said kit further comprises a detection agent specifically binding a third biomarker, said third biomarker being sFlt1, Cystatin C or MR-proADM.
21. A method for monitoring a subject, comprising:
   (a) determining the amount of DLL1, the amount of GDF-15, and optionally the amount of the third biomarker as referred to herein (i.e. sFlt1, Cystatin C or MR-proADM) in first sample of said subject and, optionally, calculating a first score based on the determined amounts,
   (b) determining the amount of DLL1, the amount of GDF-15, and optionally the amount of the third biomarker in a second sample of said subject and, optionally, calculating a second score based on the determined amounts; and comparing (c)
      c1) the first score to the second score, or
      c2) the amounts of DLL1, of GDF-15 and optionally of the third biomarker in the second sample to the amounts of DLL1, of GDF-15 and optionally of the third biomarker in the first sample, whereby the subject is monitored.
22. Use of i) a first biomarker being DLL1 and a second biomarker being GDF15, or ii) a detection agent specifically binding to said first biomarker and an detection agent specifically binding to said second biomarker for monitoring a subject.
23. The method, use, device or kit of any one of the preceding embodiments, wherein the assessment is the prediction of the risk of developing sepsis.
24. The method, use, device or kit of any one of the preceding embodiments, wherein the assessment is the prediction of the risk of developing sepsis.
25. The method, use, device or kit of any one of the preceding embodiments, wherein the assessment is the prediction of the risk that the subject's condition will deteriorate.
26. The method, use, device or kit of embodiment 25, wherein the condition of the subject deteriorates, if the subject's disease severity increases, if the subject's antibiotic therapy is intensified, if the subject is admitted to the ICU or to another unit for higher level of care, if the subject requires emergency surgery, if the subject dies in the hospital, if the subject dies within 30 days of admission, if the subject is re-hospitalized within 30 days of discharge, if the subject experiences organ dysfunction or failure, as measured e.g. with the SOFA score, and/or if the subject requires organ support.
27. The method, use, device or kit of embodiment 25 or 26, wherein the condition of the subject deteriorates, if the subject has one or more of the following outcomes: if the subject admitted to the ICU, if the subject dies in the hospital, if the subject dies within 30 days of admission, and/or if the subject is re-hospitalized within 30 days of discharge.

All references cited throughout this specification are herewith incorporated with respect to the disclosure content specifically referred to above as well as in their entireties.

### EXAMPLE 1: DETERMINATION OF BIOMARKERS

The Elecsys® Electro-ChemiLuminescence (ECL) technology and assay method is briefly described below for the determination of GDF-15. The concentration of GDF-15 was determined by a cobas e801 analyzer. Detection of GDF-15 with a cobas e801 analyzer is based on the Elecsys® Electro-ChemiLuminescence (ECL) technology. In brief, biotin-labelled and ruthenium-labelled antibodies are combined with the respective amount of undiluted sample and incubated on the analyzer. Subsequently, streptavidin-coated magnetic microparticles are added and incubated on the instrument in order to facilitate binding of the biotin-labelled immunological complexes. After this incubation step the reaction mixture is transferred into the measuring cell where the beads are magnetically captured on the surface of an electrode. ProCell M Buffer containing tripropylamine (TPA) for the subsequent ECL the reaction is then introduced into the measuring cell in order to separate bound immunoassay complexes from the free remaining particles. Induction of voltage between the working and the counter electrode then initiates the reaction leading to emission of photons by the ruthenium complexes as well as TPA. The resulting electrochemiluminescent signal is recorded by a photomultiplier and converted into numeric values indicating concentration level of the respective analyte.

DLL1 was measured using a commercially available enzyme-linked immunosorbent assay (ELISA) (RayBio® Human DLL1 ELISA Kit Catalog #: ELH-DLL1; Raybiotech, Norcross, USA). Briefly, an antibody specific for human DLL1 is coated on a 96-well microtiter plate. DLL-1 present in samples will be bound and retained by the immobilized antibody. After washing away unbound material, a second, biotinylated antibody specific for human DLL1 is pietted into the wells and binds to DLL1 present on the first antibody. After an additional washing step, horseradish peroxidase (HRP) coupled streptavidin is pipetted to the wells, and is retained in function of the amount of DLL1 present. After washing away unbound material, 3,3,5,5′-tetramethylbenzidine (TMB) is added to the wells and, in the presence of HRP, forms a reaction product, which can be measured by means of spectrophotometry.

Mid-regional proadrenomedullin (MRproADM) was measured with a commercial B.R. A. H.M.S MRproADM KRYPTOR assay, a sandwich-immunoassay which was developed for the ThermoFisher KRYPTOR platform (BRAHMS GMbH, ThermoFisher Scientific, Germany). The assay comprises an_Anti-pro-ADM sheep polyclonal antibody conjugated with europium cryptate and an Anti-pro-ADM sheep polyclonal antibody conjugated with XL665. 26 μL were used from each plasma sample and measured undiluted on a ThermoFisher KRYPTOR analyzer (ThermoFisher Scientific, Germany)

SFLT1 or sFLT-1 (Soluble fms-like tyrosine kinase-1) was measured with a commercial ECLIA assay for sFLT-1, a sandwich-immunoassay which was developed for the cobas Elecsys® ECLIA platform (ECLIA Assay from Roche Diagnostics, Germany). The assay comprises a biotinylated and a ruthenylated monoclonal antibody that specifically binds sFLT-1. 12 μL were used from each serum sample and measured undiluted on a cobas e801 analyzer (Roche Diagnostics, Germany).

CysC2 (Cystatin C) was measured with a commercial PETIA (Particle enhanced immunoturbidimetric assay) for CysC, which was developed for the cobas® clinical chemistry analyzer platforms (Roche Diagnostics, Germany). The assay comprises latex particles coated with antibodies that specifically bind CysC. Upon mixing and incubation of antibody reagent and sample, the latex enhanced particles coated with anti-cystatin C antibodies in the reagent agglutinate with the human cystatin C in the sample. The degree of the turbidity caused by the aggregate can be determined turbidimetrically at 546 nm and is proportional to the amount of cystatin C in the sample. 2 μL were used from each serum sample and measured on a cobas c 501 analyzer (Roche Diagnostics, Germany).

The NGAL (Neutrophil gelatinase-associated lipocalin) Test is a particle-enhanced turbidimetric immunoassay for the quantitative determination of NGAL 3 μL of plasma is mixed with reaction buffer R1. After a short incubation, the reaction is started by the addition of an immunoparticle suspension (polystyrene microparticles coated with mouse monoclonal antibodies to NGAL). Assay from Roche Diagnostics (Germany). NGAL in the sample causes the immunoparticles to aggregate. The degree of aggregation is quantified by the amount of light scattering measured as absorption of light. The NGAL concentration in the sample is determined by interpolation on an established calibration curve. Samples were measured on a cobas c 501 analyzer (Roche Diagnostics, Germany).

FERR was measured with a commercial ECLIA assay for Ferritin, a sandwich-immunoassay which was developed for the cobas Elecsys® ECLIA platform (ECLIA Assay from Roche Diagnostics, Germany). The assay comprises a biotinylated and a ruthenylated monoclonal antibody that specifically binds Ferritin. 10 μL were used from each serum sample and measured undiluted on a cobas e801 analyzer (Roche Diagnostics, Germany).

KL6 (KL-6) [Sialylated carbohydrate antigen KL-6]: Sialylated carbohydrate antigen KL-6 (KL-6) in samples agglutinates with mouse KL-6 monoclonal antibody coated latex through the antigen-antibody reaction. The change in absorbance caused by this agglutination is measured to determine the KL-6 level. Reagents were from Sekisui Medical Co. (Japan). 2.5 μL of Plasma were analyzed. Samples were measured on a cobas c 501 analyzer (Roche Diagnostics, Germany).

suPAR [Soluble urokinase-type plasminogen activator receptor] is a turbidimetric immunoassay that quantitatively determines suPAR in human plasma samples. The first stage of testing is an incubation of human origin specimen (EDTA or Heparin plasma) with the R1 reagent. After 5 minutes of incubation, the R2 reagent is added, and the reaction starts. The reaction buffer R2 is a suspension of latex particles coated with rat and mouse monoclonal antibodies to suPAR. After the R2 addition the process of suPAR aggregation begins, the level of accumulation is determined by the amount of scattered light during measurement of light absorption. The linear calibration curve, created before the start of the test, is used to determine the concentration of suPAR in human plasma samples. Reagents from ViroGates (Denmark). 10 μL of Plasma were analyzed. Samples were measured on a cobas c 501 analyzer (Roche Diagnostics, Germany).

LDHI2 [Lactate dehydrogenase]: UV assay Lactate dehydrogenase catalyzes the conversion of L-lactate to pyruvate; NAD is reduced to NADH in the process. L-Lactate+NAD+LDH Pyruvate+NADH+H+The initial rate of the NADH formation is directly proportional to the catalytic LDH activity. It is determined by photometrically measuring the increase in absorbance. Assay from Roche Diagnostics (Germany). 2.2 μL of Plasma were analyzed. Samples were measured on a cobas c 501 analyzer (Roche Diagnostics, Germany).

AT.pc [Antithrombin %]: Kinetic colorimetric test. This test works according to the Antithrombin (AT) Heparin Cofactor assay principle. Heparin and a predefined amount of thrombin are added to the sample in excess. All free antithrombin present binds to thrombin to form an inactive complex. Non-inhibited thrombin liberates p-nitroaniline from the chromogenic substrate MeOCO-Gly-Pro-Arg-pNA. The remaining amount of thrombin is inversely proportional to the antithrombin content of the sample and therefore the increase in absorbance at a wavelength of 415 nm can be used to calculate the antithrombin activity. Assay from Roche Diagnostics (Germany). 1 μL of Plasma were analyzed. Samples were measured on a cobas c 501 analyzer (Roche Diagnostics, Germany).

Calprotectin [Calprotectin]: The Gentian (Norway) Calprotectin Immunoassay is a Particle-Enhanced Turbidimetric Immunoassay (PETIA) for in vitro diagnostic testing of calprotectin in human plasma and serum samples. Samples were measured on a cobas c 501 analyzer (Roche Diagnostics, Germany).

### Example 2: Analysis of the Patients from the TRIAGE Study

TRIAGE Study, Kantonsspital Aarau, Switzerland, Emergency Department. (Schuetz 2013, BMC emergency medicine, 13 (1), 12).

All consecutive patients seeking ED care for non-chirurgic emergencies were included at ED admission. From a total of 4000 patients, a subset of 600 patients with infection with and without later development of sepsis; as well as 200 patients without infection were selected.

Patients with suspected infection at admission were classified into highly probable sepsis patients or infection controls according to:
Case (N=64): Highly probable sepsis cases with deterioration/higher severity within 48 h of ED presentation if they have been admitted to the ICU or meet the criteria of Rhee et al. (Rhee, C., et al. (2017). “Incidence and Trends of Sepsis in US Hospitals Using Clinical vs Claims Data, 2009-2014.” JAMA 318 (13): 1241-1249)
Control (N=207): Patients with suspected infection but no sepsis within 48 h of ED presentation

Markers were mathematically combined via logistic regression and the “area under the receiver operating characteristic” (AUC) was used as a general measure for marker performance.

In addition to the Sepsis endpoint, a “general deterioration” endpoint (i.e. whether the condition of the patient deteriorated independent from a Sepsis diagnosis) in the population of patients with suspicion of infection at ED admission was also assessed. Patients were classified in cases and controls according to:
Case: Deterioration defined as: escalation of care (i.e. admission to the ICU) or death in hospital or death within 30 days of admission or re-hospitalization within 30 days of discharge
Control: Patients with suspected infection but no Deterioration

Combinations of marker pairs (bivariate marker combinations) having improved AUCs over the single markers by at least one percentage point for the Sepsis endpoint are shown in Table 1.

TABLE 1
Bivariate marker combinations with their joint performance
(AUC.bi), the univariate performance of the first marker
(AUC.1) and the second marker (AUC.2), along with the
performance improvement of the bivariate marker over the
best single marker (Impr.AUC) for the Sepsis endpoint.

| Marker | AUC.bi | AUC.1 | AUC.2 | Impr.AUC |
|---|---|---|---|---|
| DLL1 + GDF15 | 0.8789 | 0.8038 | 0.8596 | 0.0193 |

Combinations of marker triplets (trivariate marker combinations) having improved AUCs over the bivariate marker pairs as well as all three single markers by at least one percentage point for the Sepsis endpoint are shown in Table 2.

TABLE 2
Trivariate marker combinations with their joint performance (AUC.tri),
the bivariate performance of the first two markers as listed in
Table 1 (AUC.bi), the univariate performance of the first marker
(AUC.1), the second marker (AUC.2) and the third marker (AUC.3),
along with the performance improvement of the trivariate marker
over the bivariate marker (Impr.AUC) for the Sepsis endpoint.

| Marker | AUC.tri | AUC.bi | AUC.1 | AUC.2 | AUC.3 | Impr.AUC |
|---|---|---|---|---|---|---|
| DLL1 + | 0.8999 | 0.8789 | 0.8038 | 0.8596 | 0.8481 | 0.0210 |
| GDF15 + | | | | | | |
| proADM | | | | | | |
| DLL1 + | 0.8912 | 0.8789 | 0.8038 | 0.8596 | 0.8426 | 0.0123 |
| GDF15 + | | | | | | |
| SFLT1 | | | | | | |
| DLL1 + | 0.8906 | 0.8789 | 0.8038 | 0.8596 | 0.8326 | 0.0117 |
| GDF15 + | | | | | | |
| CysC2 | | | | | | |

Combinations of marker pairs (bivariate marker combinations) having improved AUCs over the single markers by at least one percentage point for the Deterioration endpoint are shown in Table 3.

TABLE 3
Bivariate marker combinations with their joint performance
(AUC.bi), the univariate performance of the first marker
(AUC.1) and the second marker (AUC.2), along with the performance
improvement of the bivariate marker over the best single
marker (Impr.AUC) for the Deterioration endpoint.

| Marker | AUC.bi | AUC.1 | AUC.2 | Impr.AUC |
|---|---|---|---|---|
| DLL1 + GDF15 | 0.726 | 0.696 | 0.699 | 0.027 |

Examples of bivariate combinations of markers not having improved, i.e. with a negative AUC) over the single markers are shown in Table 4 for the Sepsis endpoint and Table 5 for the Deterioration endpoint. Tables 4 and 5 demonstrate the non-triviality of combining sepsis markers.

TABLE 4
Bivariate marker combinations with their joint performance (AUC.bi),
the univariate performance of the first marker (AUC.1) and the
second marker (AUC.2), along with the performance improvement
of the bivariate marker over the best single marker (Impr.AUC)
for the Sepsis endpoint. The Impr.AUC-value is negative.

| Marker | AUC.bi | AUC.1 | AUC.2 | Impr.AUC |
|---|---|---|---|---|
| DLL1 + KL6 | 0.7903 | 0.8038 | 0.5569 | −0.0136 |
| DLL1 + NGAL | 0.7957 | 0.8038 | 0.7344 | −0.0082 |
| DLL1 + suPAR | 0.7979 | 0.8038 | 0.7674 | −0.0059 |
| DLL1 + FERR | 0.8014 | 0.8038 | 0.6392 | −0.0025 |
| DLL1 + AT.pc | 0.8019 | 0.8038 | 0.5692 | −0.0019 |
| DLL1 + LDHI2 | 0.8021 | 0.8038 | 0.5740 | −0.0018 |
| DLL1 + Calprotectin | 0.8021 | 0.8038 | 0.5801 | −0.0018 |

TABLE 5
Bivariate marker combinations with their joint performance (AUC.bi),
the univariate performance of the first marker (AUC.1) and the
second marker (AUC.2), along with the performance improvement
of the bivariate marker over the best single marker Impr.AUC)
for the Deterioration endpoint. The Impr.AUC-value is negative.

| Marker | AUC.bi | AUC.1 | AUC.2 | Impr.AUC |
|---|---|---|---|---|
| DLL1 + IL6 | 0.689 | 0.696 | 0.583 | -0.007 |
| DLL1 + FERR | 0.689 | 0.696 | 0.568 | -0.007 |
| DLL1 + PENK | 0.690 | 0.696 | 0.562 | -0.006 |
| DLL1 + ALAT | 0.692 | 0.696 | 0.517 | -0.005 |
| DLL1 + CREA | 0.692 | 0.696 | 0.612 | -0.004 |
| DLL1 + NGAL | 0.693 | 0.696 | 0.645 | -0.003 |

1. A method for assessing a subject with suspected infection comprising the steps of:
(a) determining the amount of a first biomarker in a sample of the subject, said first biomarker being DLL1;
(b) determining the amount of a second biomarker in a sample of the subject, said second biomarker being GDF15;
(c) comparing the amounts of the biomarkers to references for said biomarkers and/or calculating a score for assessing the subject with suspected infection based on the amounts of the biomarkers; and
(d) assessing said subject based on the comparison and/or the calculation made in step (c).

2. The method of claim 1, wherein step (b) further comprises determining the amount of MR-proADM, sFlt1 and/or Cystatin C.

3. The method of claim 1, wherein the subject is a subject presenting at the emergency department.

4. The method of any one of claim 1, wherein the assessment is the assessment of the risk of developing sepsis and/or the assessment of the risk that the subject's condition of the subject will deteriorate.

5. The method of claim 1, wherein
a) said subject suffers from an infection or is suspected to suffer from an infection,
b) said sample is a blood sample or a sample derived therefrom, such as a blood, serum or plasma sample, and/or
c) said subject is a human.

6. (canceled)

7. (canceled)

8. A device for assessing a subject with suspected infection comprising:
(a) a measuring unit for determining the amount of a first biomarker being DLL1 and a second biomarker being GDF15 in a sample of the subject, said measuring unit comprising a detection system for the first biomarker and the second biomarker; and
(b) an evaluation unit operably linked to the measuring unit comprising a database with stored references for the first biomarker and the second biomarker as specified in claim 1 and a data processor comprising instructions for carrying out a comparison of the amount of the first biomarker and the second biomarker to references and/or for carrying out a calculation of a score for assessing the subject with suspected infection based on the amounts of the biomarkers, as specified in claim 1 and for assessing said subject based on the comparison, said evaluation unit being capable of automatically receiving values for the amounts of the biomarkers from the measuring unit.

9. The device of claim 8, wherein said measuring unit determines and comprises a detection system for a third biomarker and wherein said database comprises stored a reference for a third biomarker, said third biomarker being sFlt1, Cystatin C or MR-proADM.

10. The device of claim 8, wherein said detection system comprises at least one detection agent being capable of specifically detecting the biomarkers.

11. (canceled)

12. (canceled)

13. A kit for assessing a subject with suspected infection comprising a detection agent specifically binding to a first biomarker being DLL1 and a detection agent specifically binding to a second biomarker being GDF15, and optionally wherein said kit further comprises a detection agent specifically binding a third biomarker, said third biomarker being sFlt1, Cystatin C or MR-proADM.

14. A method for monitoring a subject, comprising:
(a) determining the amount of DLL1, the amount of GDF-15, and optionally the amount of the third biomarker selected from the group consisting of sFlt1, Cystatin C, and MR-proADM in a first sample of said subject and, optionally, calculating a first score based on the determined amounts;
(b) determining the amount of DLL1, the amount of GDF-15, and optionally the amount of the third biomarker selected from the group consisting of sFlt1, Cystatin C, and MR-proADM in a second sample of said subject and, optionally, calculating a second score based on the determined amounts; and
(c) comparing
c1) the first score to the second score, or
c2) the amounts of DLL1, of GDF-15 and optionally of the third biomarker selected from the group consisting of sFlt1, Cystatin C, and MR-proADM in the second sample to the amounts of DLL1, of GDF-15 and optionally of the third biomarker selected from the group consisting of sFlt1, Cystatin C, and MR-proADM in the first sample,
whereby the subject is monitored.

15. (canceled)

16. (canceled)

17. The kit of claim 13, wherein the assessment is the assessment of the risk of developing sepsis.

18. The method of claim 4, wherein the risk of developing sepsis within 48 hours is predicted.

19. The kit of claim 13, wherein the assessment is the prediction of the risk that the subject's condition will deteriorate.

20. The method of claim 4, wherein the condition of the subject deteriorates, if the subject's disease severity increases, if the subject's antibiotic therapy is intensified, if the subject is admitted to the ICU or to another unit for higher level of care, if the subject requires emergency surgery, if the subject dies in the hospital, if the subject dies within 30 days of admission, if the subject is re-hospitalized within 30 days of discharge, if the subject experiences organ dysfunction or failure, as measured e.g. with the SOFA score, and/or if the subject requires organ support.

21. The method of claim 4, wherein the condition of the subject deteriorates, if the subject has one or more of the following outcomes: if the subject is admitted to the ICU, if the subject dies in the hospital, if the subject dies within 30 days of admission, and/or if the subject is re-hospitalized within 30 days of discharge.

22. A method for determining the amount of a first biomarker as specified in claim 1, a second biomarker as specified in claim 1, and optionally, a third biomarker in a sample from a subject comprising the steps of:
(a) providing or obtaining the sample from the subject;
(b) determining the amount of the first biomarker in the sample of the subject;
(c) determining the amount of the second biomarker in the sample of the subject;
(d) optionally, determining the amount of the third biomarker in the sample of the subject, said third biomarker being sFlt1, Cystatin C or MR-proADM; and
(e) contacting the sample, or a portion thereof, with an agent which specifically binds the first biomarker, an agent which specifically binds the second biomarker, and, optionally, an agent which specifically binds the third biomarker.
